(19) United States
(12) Patent Application Publication
(10) Pub. No.: US 2026/0165998 A1
(43) Pub. Date: Jun. 18, 2026

(54) SYSTEMS AND METHODS FOR MONITORING FATTY ACID METABOLISM

(71) Applicants: Arizona Board of Regents on Behalf of the University of Arizona, Tucson, AZ (US); The United States Government As Represented By The Department Of Veterans Affairs, Washington, DC (US)

(72) Inventors: Kristian Doyle, Tucson, AZ (US); Rick Schnellmann, Tucson, AZ (US)

(21) Appl. No.: 19/126,133

(22) PCT Filed: Nov. 3, 2023
(86) PCT No.: PCT/US2023/036748

§ 371 Date: Apr. 30, 2025

Related U.S. Application Data
(60) Provisional application No. 63/422,082, filed on Nov. 3, 2022.

(51) Int. Cl. A61K 31/232; A61K 38/00

(52) U.S. Cl. CPC A61K 31/232; A61K 38/00

(57) ABSTRACT

The present disclosure relates to methods of determining and administering a treatment course of action. In particular, the present disclosure relates to compositions and methods for monitoring and treating neurological damage (e.g., stroke), for example, with anti-inflammation therapy.

### CROSS-REFERENCE TO RELATED APPLICATION

This application claims priority to U.S. provisional patent application Ser. No. 63/422,082, filed Nov. 3, 2022, which is incorporated herein by reference in its entirety.

### STATEMENT REGARDING FEDERALLY SPONSORED RESEARCH OR DEVELOPMENT

This invention was made with government support under Grant No. I01 RX003224 , awarded by the Veterans Administration. The government has certain rights in the invention.

### FIELD OF THE INVENTION

The present disclosure relates to methods of determining and administering a treatment course of action. In particular, the present disclosure relates to compositions and methods for monitoring and treating neurological damage.

### BACKGROUND OF THE INVENTION

According to Centers for Disease Control and Prevention, more than 795,000 people in the United States have a stroke every year. About 610,000 of these are first or new strokes. Furthermore, more than 20% of stroke survivors will have neurodegeneration that will lead to the develop of dementia within one year of their stroke.

The most significant improvements occur in the first few weeks post-stroke, often reaching a relative plateau after 3 months with less significant recovery subsequently, especially concerning motor symptoms. Thus, it is crucial for patients to have an efficient and quick access to a proper diagnostic post-stroke for assessing and monitoring damage and recovery from stroke.

### SUMMARY OF THE INVENTION

Experiments described herein identified mitochondrial fatty acid β-oxidation was substantially increased in stroked tissue at 1 day and 4 weeks following stroke. Spatial transcriptomics localized these alterations to phagocytic macrophages and microglia processing myelin lipid debris within the primary infarct and area of axonal degeneration. These results indicate that increased fatty acid oxidation in the brain is a biomarker of myelin lipid debris processing within microglia and macrophages after central nervous system (CNS) injury.

Myelin lipid processing can lead to foam cell formation in the brain and spinal cord. Foam cells form through dysregulated and overwhelmed lipid metabolism in mammalian phagocytes: lipid accumulation that exceeds the homeostatic capacity of phagocytes triggers lipid droplet formation, which results in the foamy appearance of these cells. Foam cells are associated with chronic inflammation in certain cancers and in metabolic, infectious, and autoimmune diseases. Formation of the foam cell can impair the cell's immune function and contribute to pathogenesis. For example, in atherosclerosis, foam cells derived from macrophages are critical in the initial formation, development, and instability of the atherosclerotic plaque. Foam cells that form in the brain after stroke and traumatic brain injury, and spinal cord after spinal cord injury, are dysfunctional and maladaptive for recovery.

Accordingly, provided hercin are compositions and methods for identifying and treating foam cells in the CNS after stroke and other neurological disorders that cause degenerating white matter (e.g. traumatic brain injury (TBI), spinal cord injury, multiple sclerosis (MS), progressive multifocal leukoencephalopathy (PML), leukodystrophies, Niemann-Pick disease, cerebral small vessel disease (SVD), chronic traumatic encephalopathy (CTE), neuromyelitis optica (NMO), hypoxic-ischemic injury, infections affecting the CNS like Lyme disease and HIV, radiation leukoencephalopathy, age-related white matter changes, drugs and toxins leading to leukoencephalopathy, vascular dementia, and Binswanger's disease).

For example, in some embodiments, provided herein is a method of identifying foam cells in the central nervous system (CNS) after neurological damage, comprising: a) imaging the CNs (e.g., brain or spinal cord) of a subject that has had neurological damage for the presence of brain fatty acid oxidation (BFAO) using radiopharmaceutical fatty acid metabolic probes and imaging (e.g., MRI or PET imaging); and b) identifying the subject as having foam cells in the CNS when the presence of BFAO is identified.

Further embodiments provide a method of treating a subject that has had neurological damage, comprising: a) imaging the CNS of a subject that has had neurological damage for the presence of brain fatty acid oxidation (BFAO) using radiopharmaceutical fatty acid metabolic probes and imaging; and b) administering an anti-inflammatory therapy (e.g., a cyclodextrin or other lipid chelator) when the presence of BFAO is identified.

Also provided is a method of treating a subject that has had neurological damage, comprising: a) imaging the CNS of a subject that has had neurological damage for the presence of brain fatty acid oxidation (BFAO) using radiopharmaceutical fatty acid metabolic probes and imaging; b) administering an anti-inflammatory therapy (e.g., a cyclodextrin or other lipid chelator) when the presence of BFAO is identified; and c) repeating the assaying step after the administering.

Additional embodiments provide a method of monitoring a treatment in a subject that has had neurological damage, comprising: a) imaging the CNS of a subject that has had neurological damage for the presence of brain fatty acid oxidation (BFAO) using radiopharmaceutical fatty acid metabolic probes and imaging; b) administering an anti-inflammatory therapy (e.g., a cyclodextrin or other lipid chelator) when the presence of BFAO is identified; and c) repeating the assaying step after the administering.

The present invention is not limited to particular neurological damage (e.g., due to an injury or neurological disorder). Examples include but are not limited to stroke, traumatic brain injury (TBI), spinal cord injury, multiple sclerosis (MS), progressive multifocal leukoencephalopathy (PML), leukodystrophies, Niemann-Pick disease, cerebral small vessel disease (SVD), chronic traumatic encephalopathy (CTE), neuromyelitis optica (NMO), hypoxic-ischemic injury, infections affecting the CNS like Lyme disease and HIV, radiation leukoencephalopathy, age-related white matter changes, drugs and toxins leading to leukoencephalopathy, vascular dementia, or Binswanger's disease.

The present invention is not limited to particular probes. Examples include but are not limited to ¹¹C-palmitate (CPA), ¹⁸F-palmitate derivatives, 16-¹⁸F-fluorohexadecanoic acid (FHDA), 17-¹⁸F-fluoroheptadecanoic acid (FHA), 15-(4-(2-¹⁸F fluoroethoxy)phenyl)pentadecanoic acid (F7), ¹²³I-iodophenylpentadecanoic acid (IPPA), β-methyl-1-¹¹C-heptadecanoic acid (β-Me-HA), 3-Methyl-17-¹⁸F-fluoroheptadecanoic acid (3MFHA), 5-methyl-17-¹⁸F-fluoroheptadecanoic acid (5MFHA), β-methyl-15-¹²³I-iodophenylpentadecanoic acid (β-MeIPPA), 9-^{123m}Te-telluraheptadecanoic acid (9-TeHA), 14-¹⁸F-fluoro-6-thia-heptadecanoic acid (FTHA), 16-¹⁸F-fluoro-4-thia-hexadecanoic acid (FTP), 18-¹⁸F-fluoro-4-thia-oleic acid (FTO), or trans-9(RS)-¹⁸F-fluoro-3,4(RS,RS)-methyleneheptadecanoic acid (¹⁸F-FCPHA).

In some embodiments, the method further comprises assaying a sample from said subject for the level of one or more markers selected from the group consisting of acyl carnitines and long chain fatty acids in a sample from said subject.

Certain embodiments provide the use of an anti-inflammation therapy to treat inflammation after neurological damage when the presence of BFAO is identified using radiopharmaceutical fatty acid metabolic probes and imaging.

In some embodiments, the level of one or more of said markers is increased in microglia in areas of degenerating white matter. In some embodiments, the assaying comprises assaying the brain of the subject using radionuclide and imaging. In some embodiments, the assaying comprising assaying a sample isolated from the subject (e.g., blood, a blood product, cells, or tissue). In some embodiments, the stroke is an ischemic stroke. In some embodiments, the level of itaconate, a regulator of BFAO, is determined by assaying the level of Acod1 nucleic acid or polypeptide. In some embodiments, an increased level of the markers is indicative of the presence of foam cells in the brain of the subject.

The present invention is not limited to particular anti-inflammatory therapy. For example, in some embodiments, the therapy is an agent that blocks the activity of a cytokine (e.g., IL-1α, IL-1β, and IL-1Ra, or TNF-α). The agent is, for example, a small molecule, a nucleic acid, or an antibody. Examples of anti-inflammation therapies include but are not limited to cyclodextrins, (e.g. 2-hydroxypropyl-β-cyclodextrin (HPβCD or HPbCD derivatives), anti-oxidants (e.g. edaravone, uric acid, butylphthalide, particularly dl-3-n-butylphthalide (NBP), astaxanthin, hydroxytyrosol, salidroside, sulforaphane, probucol, vitamin E (tocopherol), vitamin C (ascorbic acid), niacin, plant sterols, beta-carotene, polyphenols, curcumin, selenium, N-acetylcysteine (NAC), lycopene, alpha-lipoic acid, coenzyme Q10 (CoQ10), and green tea extract), AdipoRon, biographene quantum dots, anti-CD20 antibodies (e.g. Rituximab), anti-TREM2 antibodies, anti-CD36 antibodies, metformin, rapamycin, inhibitors of cyclic nucleotide phosphodiesterase (PDE) (e.g. Ibudilast), dimethyl fumarate, TLR antagonists and modulators, dimethyl itaconate, and neurotrophin receptor modulators (e.g. LM11A-31).

The present invention is not limited to particular intervals for assaying the levels of the disclosed markers. Examples include one or more of 24 hours, 1 week, 2 weeks, 4 weeks, 7 weeks, and 6 months (e.g., 1 day to one week; one day to 2 weeks, one day to 4 weeks, one day to 7 weeks, one day to 6 months, 1 week to 2 weeks, 1 week to 4 weeks, 1 week to 7 weeks, one week to 6 months, 2 weeks to 4 weeks, 2 weeks to 7 weeks, 2 weeks to 6 months, 4 weeks to 7weeks, 4 weeks to 6 months, or 7 weeks to 6 months, although other intervals are specifically contemplated) after stroke. In some embodiments, the assaying is repeated at an interval of, for example, daily, weekly, monthly, yearly, or another interval.

Additional embodiments are described herein.

### DESCRIPTION OF THE FIGURES

FIG. 1. Study design. Timeline indicating the time points of tissue collection after stroke or sham surgery.

FIG. 2. There is an increase in long chain fatty acids derived from myelin degeneration in the ipsilateral hemisphere for at least 4 weeks post stroke. Long chain fatty acids peaked at 4 weeks after stroke. n=5-6. The numbers in the heatmaps indicate fold change with significance tested by Welch's two-sample t test. Yellow-and red-shaded cells indicate p≤0.05 (red indicates that the mean values are significantly higher, and yellow indicates that they are significantly lower). Light red-and light-yellow shaded cells indicate 0.05<p<0.10 FIG. 3. There is an increase in acyl carnitines in the ipsilateral hemisphere for at least 4 weeks post stroke. n=5-6. The numbers in the heatmaps indicate fold change with significance tested by Welch's two-sample t test. Yellow-and red-shaded cells indicate p≤0.05 (red indicates that the mean values are significantly higher, and yellow indicates that they are significantly lower). Light red-and light-yellow shaded cells indicate 0.05<p<0.10.

FIG. 4. Schematic illustration of transportation of fatty acids into mitochondria before metabolism. After crossing the cell membrane, free fatty acids are conjugated with coenzyme-A (CoA) to form fatty acyl-CoAs, which are subsequently combined with carnitine. The resulting acyl carnitines can cross the mitochondrial membrane, after which carnitine is released, and the newly formed fatty acyl CoAs proceed to β-oxidation.

FIG. 5A-I. Changes in fatty acid oxidation localize to TMEM119 positive macrophages and microglia in the infarct (lesion) and area of axonal degeneration (ipsilateral thalamus). A-B. For spatial transcriptomics, brain sections from aged mice at 4 weeks post stroke were stained with antibodies against Tmem119 and GFAP (A.), or NeuN and GFAP (B.). Gene expression data was gathered from cells expressing these markers from ipsilateral cortex (CX), hippocampus (HC), thalamus (TH), and infarct (Lesion), and corresponding areas from the contralateral side, n=4. Scale bars 1000 μm. C-D. To confirm cell specificity the expression of genes that are traditionally associated with astrocytes and microglia (C.), and neurons and astrocytes (D.) was assessed in the transcripts isolated from each brain region using each marker. E-G. The overall expression of genes related to glycolysis is higher in neurons and microglia than that of genes related to fatty acid oxidation, whereas astrocytes express more genes related to fatty acid oxidation than those related to glycolysis. Except for a modest decrease in glycolysis related genes in the ipsilateral thalamus, neuronal expression of these genes did not differ between the ipsi-and contralateral sides (E). In the ipsilateral hippocampus and thalamus, astrocytes reduced the expression of genes associated with fatty acid oxidation. (F). Microglia in the ipsilateral cortex and thalamus displayed a marked reduction in the expression of genes linked to glycolysis, which was accompanied by an increase in genes related to fatty acid oxidation (shown in red rectangle) (G). There was also a marked increase in the expression of genes related to fatty acid oxidation in microglia and in macrophages in the infarct (H).

Macrophages and microglia in the infarct and area of axonal degeneration take on a foamy appearance in the weeks after stroke due to the accumulation of lipid droplets derived from the uptake of myelin debris (I).

FIG. 6A-C. Delivery of a ¹⁸F-palmitate derivative detects fatty acid metabolism in the heart and illuminates the area of infarction in the brain 2 weeks following stroke. (A.) Structure of the ¹⁸F-palmitate derivative. (B.) The ¹⁸F-palmitate derivative accumulates in the heart but not the brain in naïve mice. (C.) The ¹⁸F-palmitate derivative accumulates in the infarct in a stroked mouse at 2 weeks following stroke.

### DEFINITIONS

To facilitate an understanding of the present invention, a number of terms and phrases are defined below:

As used herein, the terms “detect”, “detecting” or “detection” may describe either the general act of discovering or discerning or the specific observation of a detectably labeled composition.

As used herein, the term “foam cell” refers to a lipid-laden macrophage or glial cell characterized by its distinctive ‘foamy’ cytoplasmic appearance due to the accumulation of lipid droplets. In some instances, these cells arise due to the phagocytosis of myelin, cholesterol, and other lipids and are commonly associated with various neurodegenerative conditions and inflammatory responses within the CNS.

As used herein, the term “subject” refers to any organisms that are screened using the diagnostic methods described herein. Such organisms preferably include, but are not limited to, mammals (e.g., humans).

The term “diagnosed,” as used herein, refers to the recognition of a disease by its signs and symptoms, or genetic analysis, pathological analysis, histological analysis, and the like.

As used herein, the term “nucleic acid molecule” refers to any nucleic acid containing molecule, including but not limited to, DNA or RNA. The term encompasses sequences that include any of the known base analogs of DNA and RNA including, but not limited to, 4-acctylcytosine, 8-hydroxy-N6-methyladenosine, aziridinylcytosine, pseudoisocytosinc, 5-(carboxyhydroxylmethyl) uracil, 5-fluorouracil, 5-bromouracil, 5-carboxymethylaminomethyl-2-thiouracil, 5-carboxymethylaminomethyluracil, dihydrouracil, inosine, N6-isopentenyladenine, 1-methyladenine, 1-methylpseudouracil, 1-methylguanine, 1-methylinosine, 2,2-dimethylguanine, 2-methyladenine, 2-methylguanine, 3-methylcytosine, 5-methylcytosine, N 6-methyladenine, 7-methylguanine, 5-methylaminomethyluracil, 5-methoxy-aminomethyl-2-thiouracil, beta-D-mannosylqueosine, 5′-methoxycarbonylmethyluracil, 5-methoxyuracil, 2-methylthio-N6-isopentenyladenine, uracil- 5-oxyacetic acid methylester, uracil- 5-oxyacetic acid, oxybutoxosine, pseudouracil, queosine, 2-thiocytosine, 5-methyl-2-thiouracil, 2-thiouracil, 4-thiouracil, 5-methyluracil, N-uracil- 5-oxyacetic acid methylester, uracil- 5-oxyacetic acid, pseudouracil, queosine, 2-thiocytosine, and 2,6-diaminopurine.

The term “gene” refers to a nucleic acid (e.g., DNA) sequence that comprises coding sequences necessary for the production of a polypeptide, precursor, or RNA (e.g., rRNA, tRNA). The polypeptide can be encoded by a full-length coding sequence or by any portion of the coding sequence so long as the desired activity or functional properties (e.g., enzymatic activity, ligand binding, signal transduction, immunogenicity, etc.) of the full-length or fragments are retained.

The term also encompasses the coding region of a structural gene and the sequences located adjacent to the coding region on both the 5′ and 3′ ends for a distance of about 1 kb or more on either end such that the gene corresponds to the length of the full-length mRNA. Sequences located 5′ of the coding region and present on the mRNA are referred to as 5′ non-translated sequences. Sequences located 3′ or downstream of the coding region and present on the mRNA are referred to as 3′ non-translated sequences. The term “gene” encompasses both cDNA and genomic forms of a gene. A genomic form or clone of a gene contains the coding region interrupted with non-coding sequences termed “introns” or “intervening regions” or “intervening sequences.” Introns are segments of a gene that are transcribed into nuclear RNA (hnRNA); introns may contain regulatory elements such as enhancers. Introns are removed or “spliced out” from the nuclear or primary transcript; introns therefore are absent in the messenger RNA (mRNA) transcript. The mRNA functions during translation to specify the sequence or order of amino acids in a nascent polypeptide.

As used herein, the term “oligonucleotide,” refers to a short length of single-stranded polynucleotide chain. Oligonucleotides are typically less than 200 residues long (e.g., between 15 and 100), however, as used herein, the term is also intended to encompass longer polynucleotide chains. Oligonucleotides are often referred to by their length. For example, a 24 residue oligonucleotide is referred to as a “24-mer”. Oligonucleotides can form secondary and tertiary structures by self-hybridizing or by hybridizing to other polynucleotides. Such structures can include, but are not limited to, duplexes, hairpins, cruciforms, bends, and triplexes.

The term “isolated” when used in relation to a nucleic acid, as in “an isolated oligonucleotide” or “isolated polynucleotide” refers to a nucleic acid sequence that is identified and separated from at least one component or contaminant with which it is ordinarily associated in its natural source. Isolated nucleic acid is such present in a form or setting that is different from that in which it is found in nature. In contrast, non-isolated nucleic acids as nucleic acids such as DNA and RNA found in the state they exist in nature. For example, a given DNA sequence (e.g., a gene) is found on the host cell chromosome in proximity to neighboring genes; RNA sequences, such as a specific mRNA sequence encoding a specific protein, are found in the cell as a mixture with numerous other mRNAs that encode a multitude of proteins. However, isolated nucleic acid encoding a given protein includes, by way of example, such nucleic acid in cells ordinarily expressing the given protein where the nucleic acid is in a chromosomal location different from that of natural cells, or is otherwise flanked by a different nucleic acid sequence than that found in nature. The isolated nucleic acid, oligonucleotide, or polynucleotide may be present in single-stranded or double-stranded form. When an isolated nucleic acid, oligonucleotide or polynucleotide is to be utilized to express a protein, the oligonucleotide or polynucleotide will contain at a minimum the sense or coding strand (i.e., the oligonucleotide or polynucleotide may be single-stranded), but may contain both the sense and anti-sense strands (i.e., the oligonucleotide or polynucleotide may be double-stranded).

“Mass Spectrometry” (MS) is a technique for measuring and analyzing molecules that involves fragmenting a target molecule, then analyzing the fragments, based on their mass/charge ratios, to produce a mass spectrum that serves as a “molecular fingerprint”. Determining the mass/charge ratio of an object is done through means of determining the wavelengths at which electromagnetic energy is absorbed by that object. There are several commonly used methods to determine the mass to charge ration of an ion, some measuring the interaction of the ion trajectory with electromagnetic waves, others measuring the time an ion takes to travel a given distance, or a combination of both. The data from these fragment mass measurements can be searched against databases to obtain definitive identifications of target molecules. Mass spectrometry is also widely used in other areas of chemistry, like petrochemistry or pharmaceutical quality control, among many others.

The term “metabolism” refers to the chemical changes that occur within the tissues of an organism, including “anabolism” and “catabolism”. Anabolism refers to biosynthesis or the buildup of molecules and catabolismrefers to the breakdown of molecules.

A “metabolite” is an intermediate or product resulting from metabolism. Metabolites are often referred to as “small molecules”.

The term “metabolomics” refers to the study of cellular metabolites.

As used herein, the term “sample” is used in its broadest sense. In one sense, it is meant to include a specimen or culture obtained from any source, as well as biological and environmental samples. Biological samples may be obtained from animals (including humans) and encompass fluids, solids, tissues (e.g., biopsy samples), cells, and gases. Biological samples include blood products, such as plasma, serum and the like. Such examples are not however to be construed as limiting the sample types applicable to the present invention.

### DETAILED DESCRIPTION OF THE INVENTION

Inflammation is a crucial part of the healing process after a CNS injury such as a stroke and is required to restore tissue homeostasis. However, the inflammatory response to CNS injuries also worsens neurodegeneration and creates a tissue environment that is unfavorable to regeneration for several months, thereby postponing recovery. It can also contribute to the development of dementia.

Microglia and macrophages that take on a foamy appearance are one of the most prominent immune cell types within lesions and areas of degenerating white matter in the CNS. Foam cells form as a result of mechanisms of myelin lipid clearance becoming overwhelmed, and that they are a key driver of chronic inflammation in CNS diseases. Therefore, imaging lipid accumulation and metabolism in microglia and macrophages is a promising biomarker for visualizing these cells as they transition into foam cells.

Accordingly, experiments described herein evaluated how lipid metabolism is altered in areas of foam cell accumulation after stroke. Stroke was selected as the primary means to induce a lesion in the CNS. This choice was informed by the significant damage strokes cause, leading to the rapid formation of foam cells in the discernible, focal region of the infarct shortly after the event. However, the present invention is not limited to applications related to stroke.

In experiments described herein, the brain metabolomes of the ipsilateral and contralateral hemispheres in aged male mice up to 12 weeks post-stroke were compared with those of age-matched naïve and sham mice. Spatial transcriptomics was used to ascertain whether changes in lipid metabolism could be localized to microglia and macrophages exhibiting a foamy appearance. The findings revealed elevated mitochondrial fatty acid β-oxidation, as indicated by increased fatty acid and acyl carnitine levels, in microglia and macrophages within the stroked tissue for at least 4 weeks after the stroke.

For proof of concept, an ¹⁸F-palmitate derivative was administered to mice two weeks post-stroke to ascertain if increased fatty acid β-oxidation could be detected in stroke-affected tissue via PET MRI. The tracer highlighted fatty acid β-oxidation in the heart tissue of naïve mice and showed no such activity in the brain tissue of these mice. This was expected because the heart typically metabolizes fatty acids for fuel, while the brain predominantly relies on glucose. However, two weeks after the stroke, the tracer indicated heightened fatty acid β-oxidation in the infarcted tissue of a stroked mouse.

Ischemic strokes are caused by the blockage or narrowing of a brain blood vessel which results in insufficient delivery of oxygen and glucose to support cellular homeostasis. Although this acute perturbation in brain metabolism is well characterized, little is known about how brain metabolism is altered in the weeks and months during recovery. This is an important knowledge gap to address because, although cells adapt to their environment by undergoing metabolic reprogramming, metabolic reprogramming also alters cellular function (Sun L, Yang X, Yuan Z, et al. Metabolic Reprogramming in Immune Response and Tissue Inflammation. Arterioscler Thromb Vasc Biol 2020; 40:1990-2001). Therefore, knowing more about metabolic changes in the brain in the chronic period after injury such as stroke may reveal new targets for intervention. Furthermore, long-lasting metabolic abnormalities in the brain may be amenable for diagnostic neuroimaging.

For example, in some embodiments, provided herein is a method of identifying foam cells in the CNS (e.g., brain or spinal cord) after neurological damage, comprising: a) imaging the CNS of a subject that has had neurological damage for the presence of brain fatty acid oxidation (BFAO) using radiopharmaceutical fatty acid metabolic probes and imaging (e.g., MRI or PET imaging); and b) identifying the subject as having foam cells in the CNS when the presence of BFAO is identified.

Further embodiments provide a method of treating a subject that has had neurological damage, comprising: a) imaging the CNS of a subject that has had neurological damage for the presence of brain fatty acid oxidation (BFAO) using radiopharmaceutical fatty acid metabolic probes and imaging; and b) administering an anti-inflammatory therapy (e.g., a cyclodextrin or other lipid chelator) when the presence of BFAO is identified.

Also provided is a method of treating a subject that has had neurological damage, comprising: a) imaging the CNS of a subject that has had neurological damage for the presence of brain fatty acid oxidation (BFAO) using radiopharmaceutical fatty acid metabolic probes and imaging; b) administering an anti-inflammatory therapy (e.g., a cyclodextrin or other lipid chelator) when the presence of BFAO is identified; and c) repeating the assaying step after the administering.

Additional embodiments provide a method of monitoring a treatment in a subject that has had neurological damage, comprising: a) imaging the CNS of a subject that has had neurological damage for the presence of brain fatty acid oxidation (BFAO) using radiopharmaceutical fatty acid metabolic probes and imaging; b) administering an anti-inflammatory therapy (e.g., a cyclodextrin or other lipid chelator) when the presence of BFAO is identified; and c) repeating the assaying step after the administering.

The present invention is not limited to particular neurological damage (e.g., due to an injury or neurological disorder). Examples include but are not limited to stroke, traumatic brain injury (TBI), spinal cord injury, multiple sclerosis (MS), progressive multifocal leukoencephalopathy (PML), leukodystrophies, Niemann-Pick disease, cerebral small vessel disease (SVD), chronic traumatic encephalopathy (CTE), neuromyelitis optica (NMO), hypoxic-ischemic injury, infections affecting the CNS like Lyme disease and HIV, radiation leukoencephalopathy, age-related white matter changes, drugs and toxins leading to leukoencephalopathy, vascular dementia, or Binswanger's disease.

The present invention is not limited to particular probes. Examples include but are not limited to ¹¹C-palmitate (CPA), ¹⁸F-palmitate derivatives, 16-¹⁸F-fluorohexadecanoic acid (FHDA), 17-¹⁸F-fluoroheptadecanoic acid (FHA), 15-(4-(2-¹⁸F fluoroethoxy)phenyl)pentadecanoic acid (F7), ¹²³I-iodophenylpentadecanoic acid (IPPA), β-methyl-1-11C-heptadecanoic acid (B-Me-HA), 3-Methyl-17-¹⁸F-fluoroheptadecanoic acid (3MFHA), 5-methyl-17-¹⁸F-fluoroheptadecanoic acid (5MFHA), β-methyl- 15-¹²³I-iodophenylpentadecanoic acid (β-MeIPPA), 9-^{123m}Te-telluraheptadecanoic acid (9-TeHA), 14-¹⁸F-fluoro-6-thia-heptadecanoic acid (FTHA), 16-¹⁸F-fluoro-4-thia-hexadecanoic acid (FTP), 18-¹⁸F-fluoro-4-thia-oleic acid (FTO), or trans-9(RS)-¹⁸F-fluoro-3,4(RS, RS)-methyleneheptadecanoic acid (¹⁸F-FCPHA) (See e.g., Mather K J and DeGrado T. Imaging of Myocardial Fatty Acid Oxidation. Biochim Biophys Acta 2017; 1860(10): 1535-1543; herein incorporated by reference in its entirety).

In some embodiments, an anti-inflammation therapy is administered to the subject when the presence of BFAO is identified. In some embodiments, levels of BFAO is monitored after stroke and/or during anti-inflammation therapy (e.g., at regular intervals of daily, weekly, monthly, yearly, or other interval) to monitor inflammation in the subject. The levels of the markers can be used to determine a treatment course of action (e.g., starting, stopping, or changing anti-inflammation therapy).

The present invention is not limited to a particular anti-inflammatory therapy. For example, in some embodiments, the therapy is an agent that blocks the activity of a cytokine (e.g., IL-1α, IL-1β, and IL-1Ra, or TNF-α). The agent is, for example, a small molecule, a nucleic acid, or an antibody. Examples of anti-inflammation therapies used after stroke include but are not limited to, hydroxypropyl-β-cyclodextrin (HPβCD) and HPβCD derivatives, AdipoRon, biographene quantum dots, anti-CD20 antibodies (e.g. Rituximab), anti-TREM2 antibodies, anti-CD36 antibodies, metformin, rapamycin, inhibitors of cyclic nucleotide phosphodiesterase (PDE) (e.g. Ibudilast), dimethyl fumarate, TLR antagonists and modulators, dimethyl itaconate, and neurotrophin receptor modulators (e.g. LM11A-31).

In some embodiments, the cyclodextrin is, for example, 2-Hydroxypropyl-b-cyclodextrin (HPbCD) (See e.g., Becktel et al., The Journal of Neuroscience, Jan. 12, 2022 42(2):325-348 325; herein incorporated by reference in its entirety). In some embodiments, the cyclodextrin is a cyclodextrin described in WO 00/04888; herein incorporated by reference in its entirety) is utilized.

Therapies may be delivered using any suitable method (e.g., oral administration, intravenous (IV) injection, subcutaneous injection, intramuscular injection, intranasal delivery, intra-arterial infusion, intrathecal administration, or transdermal patches).

The present disclosure is not limited to particular values for a reference level of the markers. In some embodiments, the reference level is the level of the markers in a subject that has not had a stroke. In some embodiments, the reference level is an average of a given population of cells or samples obtained from a representative number of patients. In some embodiments, the reference level is pre-set. In some embodiments, the reference level is based on the level of expression of the markers or in the patient (e.g., the level at the beginning of treatment).

The present invention is not limited to stroke. The compositions and methods described herein find use in monitoring and treating any number of neurological disorders that cause degenerating white matter and the accumulation of foam cells in the CNS (e.g. traumatic brain injury (TBI), spinal cord injury, multiple sclerosis (MS), progressive multifocal leukoencephalopathy (PML), leukodystrophies, Niemann-Pick disease, cerebral small vessel disease (SVD), chronic traumatic encephalopathy (CTE), neuromyelitis optica (NMO), hypoxic-ischemic injury, infections affecting the CNS like Lyme disease and HIV, radiation leukoencephalopathy, age-related white matter changes, drugs and toxins leading to leukoencephalopathy, vascular dementia, and Binswanger's disease).

The present disclosure is not limited to particular methods of measuring the level of the described markers. In some embodiments, markers are identified using in vivo imaging methods. In some embodiments, markers are identified in a sample (e.g., blood or tissue).

Metabolites may be detected using any suitable method including, but not limited to, liquid and gas phase chromatography, alone or coupled to mass spectrometry (See e.g., experimental section below), NMR (See e.g., US patent publication 20070055456, herein incorporated by reference), immunoassays, chemical assays, spectroscopy and the like. In some embodiments, commercial systems for chromatography and NMR analysis are utilized.

In other embodiments, metabolites (e.g., biomarkers and derivatives thereof) are detected using optical imaging techniques such as magnetic resonance spectroscopy (MRS), magnetic resonance imaging (MRI), Positron emission tomography (PET), CAT scans, ultrasound, MS-based tissue imaging or X-ray detection methods(e.g., energy dispersive x-ray fluorescence detection).

Any suitable method may be used to analyze the biological sample in order to determine the presence, absence or level(s) of the one or more metabolites in the sample. Suitable methods include chromatography (e.g., HPLC, gas chromatography, liquid chromatography), mass spectrometry (e.g., MS, MS-MS), enzyme-linked immunosorbent assay (ELISA), antibody linkage, other immunochemical techniques, biochemical or enzymatic reactions or assays, and combinations thereof. Further, the level(s) of the one or more metabolites may be measured indirectly, for example, by using an assay that measures the level of a compound (or compounds) that correlates with the level of the biomarker(s) that are desired to be measured.

In embodiment of the present invention that utilize probes to detect BFAO, any suitable imaging method may be utilized (e.g., MRI or PET scans) to visualize the probe.

In some embodiments, a computer-based analysis program is used to translate the raw data generated by the detection assay (e.g., the presence, absence, or amount of a given marker or markers) into data of predictive value for a clinician. The clinician can access the predictive data using any suitable means. Thus, in some preferred embodiments, the present invention provides the further benefit that the clinician, who is not likely to be trained in genetics or molecular biology, need not understand the raw data. The data is presented directly to the clinician in its most useful form. The clinician is then able to immediately utilize the information in order to optimize the care of the subject.

The present invention contemplates any method capable of receiving, processing, and transmitting the information to and from laboratories conducting the assays, information provides, medical personal, and subjects. For example, in some embodiments of the present invention, a sample (e.g., a blood or blood product sample) is obtained from a subject and submitted to a profiling service (e.g., clinical lab at a medical facility, genomic profiling business, etc.), located in any part of the world (e.g., in a country different than the country where the subject resides or where the information is ultimately used) to generate raw data.

Where the sample comprises a tissue or other biological sample, the subject may visit a medical center to have the sample obtained and sent to the profiling center, or subjects may collect the sample themselves and directly send it to a profiling center. Where the sample comprises previously determined biological information, the information may be directly sent to the profiling service by the subject (e.g., an information card containing the information may be scanned by a computer and the data transmitted to a computer of the profiling center using an electronic communication systems). Once received by the profiling service, the sample is processed and a profile is produced (i.e., presence of BFAO), specific for the information desired for the subject.

The profile data is then prepared in a format suitable for interpretation by a treating clinician. For example, rather than providing raw expression data, the prepared format may represent an assay result (e.g., presence of BFAO) for the subject, along with recommendations for particular treatment options. The data may be displayed to the clinician by any suitable method. For example, in some embodiments, the profiling service generates a report that can be printed for the clinician (e.g., at the point of care) or displayed to the clinician on a computer monitor.

In some embodiments, the information is first analyzed at the point of care or at a regional facility. The raw data is then sent to a central processing facility for further analysis and/or to convert the raw data to information useful for a clinician or patient. The central processing facility provides the advantage of privacy (all data is stored in a central facility with uniform security protocols), speed, and uniformity of data analysis. The central processing facility can then control the fate of the data following treatment of the subject. For example, using an electronic communication system, the central facility can provide data to the clinician, the subject, or researchers.

In some embodiments, the subject is able to directly access the data using the electronic communication system. The subject may choose further intervention or counseling based on the results. In some embodiments, the data is used for research use. For example, the data may be used to further optimize the inclusion or elimination of markers as useful indicators of a particular condition or stage of disease or as a companion diagnostic to determine a treatment course of action.

Compositions for use in the screening, diagnostic, prognostic, and therapeutic methods described herein include, but are not limited to, probes, amplification oligonucleotides, and the like. In some embodiments, compositions are provided in the form of a kit. In some embodiments, kits include all components necessary, sufficient or useful for detecting the markers described herein (e.g., reagents, controls, instructions, etc.). The kits described herein find use in research, therapeutic, screening, and clinical applications.

### EXPERIMENTAL

The following examples are provided in order to demonstrate and further illustrate certain preferred embodiments and aspects of the present invention and are not to be construed as limiting the scope thereof.

### Example 1

### Materials and Methods

### Animals

Sample sizes for the study were determined using power calculation with G*Power software (version 3.1.9.7, Heinrich Heine Universität, Düsseldorf, Germany; downloaded from www.psychologie.hhu.de/arbeitsgruppen/allgemeine-psychologie-und-arbeitspsychologie/gpower.html) (Faul F, Erdfelder E, Lang A-G, et al. G*Power 3: A flexible statistical power analysis program for the social, behavioral and biomedical sciences. Behav Res Methods 2007; 39:175-191). With an effect size (based on estimated group means and standard deviations (SDs) from previous experiments) of 2.5, α 0.05 and power 90%, the group size was determined to be 5. Due to stroke surgery causing increased mortality in aged animals, group sizes were increased by 1-2 animals as necessary, to ensure that the final n would be 5 in each group. In total, 86 male C57BL/6J mice, sourced from the aged rodent colony at the National Institute on Aging, were used in this study. Six of them were young adults (7-month-old), and 80 were aged (18-to 20-month-old). The young mice were sacrificed without undergoing stroke or sham surgery (young naïve group). Four of the aged mice were stroked and sacrificed at 4 weeks post stroke for spatial transcriptomics. The remaining 76 mice were divided randomly (using GraphPad Prism Quick Calcs) into the following three groups: aged naïve (n=6), aged sham (n=30), and aged stroke (n=40). The aged stroke and sham mice were sacrificed at 1 day post ischemia (dpi) (7 stroke and 6 sham) and 2 weeks (7 stroke and 6 sham), 4 weeks (7 stroke and 6 sham), 8 weeks (6 stroke and 6 sham), and 12 weeks (6 stroke and 6 sham) after stroke or sham surgery. The pre-set exclusion criteria of the study were (1) unsuccessful induction of ischemia (4 mice), (2) death of the animal during the experiment (3 mice), and (3) being a statistically significant outlier in any of the analyses (4 mice). All animal experiments followed the NIH guidelines and were approved by the University of Arizona Institutional Animal Care and Use Committee. RIGOR criteria and ARRIVE guidelines were followed when conducting and reporting the experiments.^{8,9} Every effort was made to minimize the harm and suffering of the animals.

### Stroke and Sham Surgeries

The permanent distal middle cerebral artery occlusion (dMCAO) was carried out as described previously (Doyle K P, Fathali N, Siddiqui M R, et al. Distal hypoxic stroke: a new mouse model of stroke with high throughput, low variability and a quantifiable functional deficit. J Neurosci Methods 2012; 207:31-40). Briefly, anesthesia was induced with 3% isoflurane (JD Medical, Phoenix, AZ, USA), and maintained throughout surgery. After the temporalis muscle was exposed and retracted, a hole (1 mm in diameter) was drilled into the temporal bone to expose the MCA. The dura was removed, after which the artery was cauterized. The temporalis muscle was replaced, and the wound closed using surgical glue. Body temperature of the animals was maintained at 37±0.5° C. during the procedure using a heating pad equipped with a rectal probe. Respiration and temperature were monitored throughout surgery. Immediately after surgery, mice were placed in a hypoxia chamber (Coy Laboratory products, Grass Lake, MI, USA) containing 9% oxygen and 91% nitrogen for 45 minutes. The purpose of hypoxia in this model is to both increase infarct size and reduce variability in infarct size (Doyle et al., 2012, supra). A single dose of buprenorphine hydrochloride (Buprenex® Injection 0.3 mg/mL, Henry Schein Medical, Melville, NY, USA; 0.1 mg/kg s.c.) was administered prior to surgery, and sustained release buprenorphine (Buprenorphine SR 1 mg/mL, Zoopharm LLC, Laramie, WY, USA; 1 mg/kg s.c.) was administered 24 hours after surgery as a post-operative analgesic. The same steps, including 45 minutes of hypoxia, were performed on sham operated animals, except for cauterizing the MCA.

### Perfusion and Tissue Collection

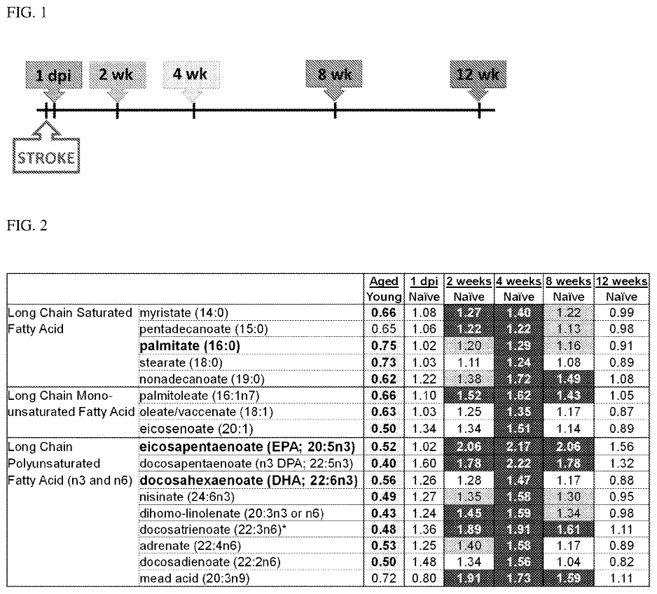

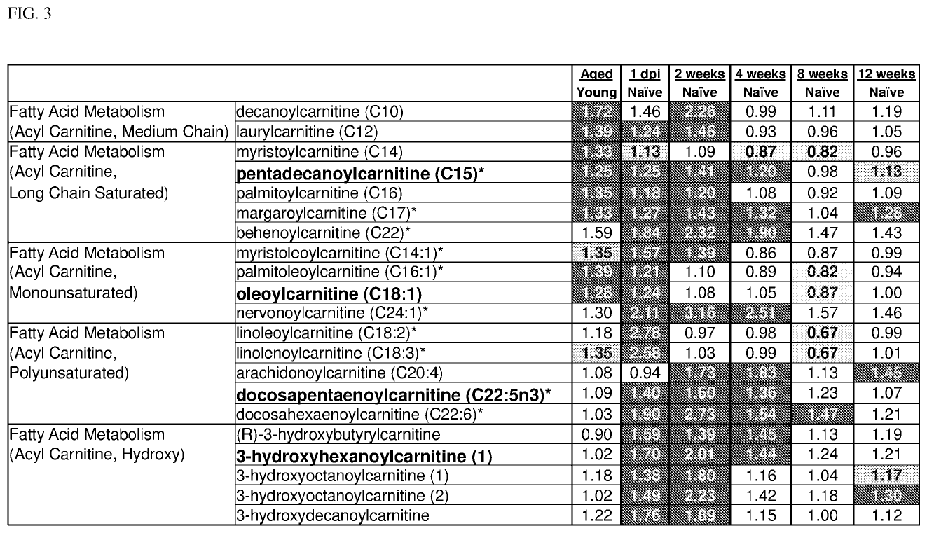

At the time points described in FIG. 1, mice were anesthetized with 3% isoflurane. Blood samples were collected from the heart, after which mice were transcardially perfused with 0.9% saline solution. For the spatial transcriptomics, whole brains were carefully removed and placed immediately into 4% PFA in PBS for 22 hours at 4° C., after which they were transferred to 30% sucrose solution at 4° C. for 48 hours, embedded in paraffin, and cut into 7 μm thick sections. For the metabolomics contralateral and ipsilateral hemispheres were dissected, snap frozen in liquid nitrogen, and shipped to Metabolon Inc. (Morrisville, NC, USA) for metabolomic analysis. The tissue was not further separated into smaller brain regions due to a minimum requirement of 50 mg of tissue outlined in Metabolon's global metabolic profiling protocol. A guide to the dissection is provided in FIG. 2.

### Metabolomic Analysis

Global metabolite profiling analysis on brain tissue was performed by Metabolon Inc. using ultra high-performance liquid chromatography coupled to tandem mass spectrometry (UPLC-MS/MS). In brief, following receipt by Metabolon, samples were inventoried and immediately stored at −80° C. Samples were prepared using the automated MicroLab STAR® system from Hamilton Company (Reno, NV/Franklin, MA, USA). Several recovery standards were added prior to the first step in the extraction process for quality control (QC) purposes. To remove protein, dissociated small molecules bound to protein or trapped in the precipitated protein matrix, and to recover chemically diverse metabolites, proteins were precipitated with methanol under vigorous shaking for 2 min (GenoGrinder 2000; Glen Mills, Clifton, NJ, USA) followed by centrifugation. The resulting extract was divided into five fractions: two for analysis by two separate reverse phase (RP)/UPLC-MS/MS methods with positive ion mode electrospray ionization (ESI), one for analysis by RP/UPLC-MS/MS with negative ion mode ESI, one for analysis by HILIC/UPLC-MS/MS with negative ion mode ESI, and one reserved for backup. Samples were placed briefly on a Turbo Vap® (Zymark, Biotage, Uppsala, Sweden) to remove the organic solvent. The sample extracts were stored overnight under nitrogen before preparation for analysis.

Several types of controls were analyzed in concert with the experimental samples: a pooled matrix sample generated by taking a small volume of each experimental sample served as a technical replicate throughout the data set; extracted water samples served as process blanks; and a cocktail of QC standards that were carefully chosen not to interfere with the measurement of endogenous compounds were spiked into every analyzed sample, allowing instrument performance monitoring, and aiding chromatographic alignment.

Instrument variability was determined by calculating the median relative standard deviation (RSD) for the standards that were added to each sample prior to injection into the mass spectrometers. Overall process variability was determined by calculating the median RSD for all endogenous metabolites (non-instrument standards) present in 100% of the pooled matrix samples. Experimental samples were randomized across the platform run with QC samples spaced evenly.

For UPLC-MS/MS, all methods utilized a Waters ACQUITY ultra-performance liquid chromatography (UPLC) and a Thermo Scientific Q-Exactive high resolution/accurate mass spectrometer interfaced with a heated electrospray ionization (HESI-II) source and Orbitrap mass analyzer operated at 35,000 mass resolution. The sample extract was dried then reconstituted in solvents compatible to each of the four methods.

For data extraction and compound identification, raw data was extracted, peak-identified and QC processed using Metabolon's hardware and software. Compounds were identified by comparison to library entries of purified standards or recurrent unknown entities. Metabolon maintains a library based on authenticated standards that contains the retention time/index (RI), mass to charge ratio (m/z), and chromatographic data (including MS/MS spectral data) on all molecules present in the library. Furthermore, biochemical identifications were based on three criteria: retention index within a narrow RI window of the proposed identification, accurate mass match to the library +/−10 ppm, and the MS/MS forward and reverse scores between the experimental data and authentic standards.

### Spatial Transcriptomics

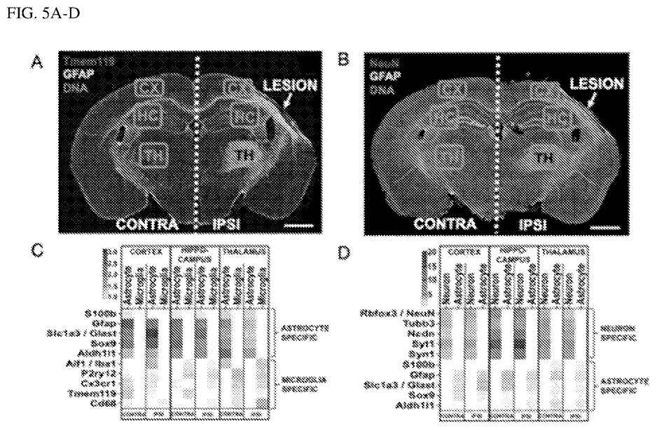

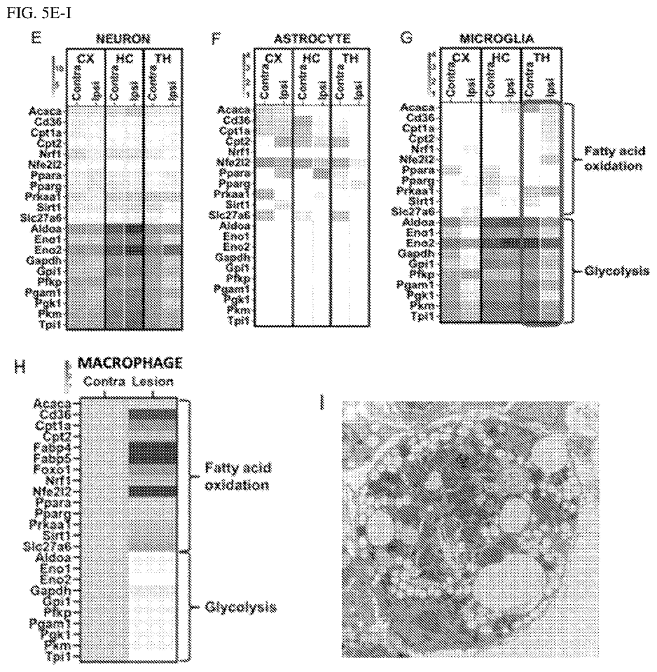

Spatial transcriptomics was completed at NanoString Technologies (Seattle, WA, USA). After arrival, the FFPE mouse brain sections were baked for 1 hr at 65° C., after which they were processed on a Leica automation platform with a protocol that includes three major steps: 1) slide baking, 2) Antigen Retrieval for 20 min at 100° C., 3) 0.1 μg/ml Proteinase K treatment for 15 min. Slides were then incubated with GeoMx WTA assay probe cocktail overnight. On the following day the slides were washed and incubated with fluorescent anti-Tmem119 and anti-GFAP antibodies or anti-NeuN and anti-GFAP antibodies barcoded with photocleavable oligonucleotide tagged RNA probes (FIG. 5 A-B) before loading onto a Nanostring GeoMx Mouse Whole Transcriptome Atlas Digital Profiler. The slides were fluorescently scanned, and regions of interest (ROI) were selected (FIG. 5 A-B). The ROIs were segmented using the fluorescent antibodies so that the transcriptomes of Tmem119+microglia, GFAP+astrocytes, and NeuN+neurons could be collected separately. Sequencing was completed on an Illumina NGS platform as previously described (Zollinger DR, Lingle SE, Sorg K, et al. GeoMxTM RNA Assay: High Multiplex, Digital, Spatial Analysis of RNA in FFPE Tissue. Methods Mol Biol 2020; 2148:331-345).

### PET MRI

The 18F-palmitate derivative was synthesized by introducing a sulfur atom to the palmitate structure, a method designed to metabolically trap the radiotracer, enhancing its retention in metabolic pathways. Both naïve mice and those two weeks post-stroke, were used, with all animals being cared for in line with specific institutional guidelines. After intravenous injection of the synthesized 18F-palmitate derivative, the distribution and accumulation of the radiotracer were observed by positron emission tomography (PET) imaging focusing on heart tissue in naïve mice due to its natural fatty acid metabolism affinity and on the infarcted brain region in post-stroke mice.

### Statistics

Median-scaled raw data was used to generate the heat maps in FIGS. 2 and 3. Welch's two-sample t-test was used to test whether two unknown means were different between two independent populations. The heat maps for FIG. 5 were generated by using the negative normalized (mean+1 standard deviation (SD)) gene expression data from each cell type and brain area. Statistical analyses were performed using GraphPad Prism software 9.3.1 (GraphPad Software, LaJolla, CA, USA), and normality was assessed using the Kolmogorov-Smirnov test. Statistical tests and sample sizes are provided in each figure legend, and p values less than 0.05 were considered to be statistically significant. Statistically significant outliers, calculated using GraphPad Prism QuickCalcs, were excluded from the datasets. Data are presented as mean+SD.

### Results

### Overview of the Analysis

The timeline of the experiment is presented in FIG. 1. Mice underwent stroke or sham surgery and were sacrificed at the indicated timepoints. The ipsilateral and contralateral hemispheres were then dissected and used for the metabolomic evaluation. Equivalent brain regions from young naïve mice, aged naïve mice, and sham mice were dissected and used for metabolomic analysis as reference controls.

The total number of biochemicals detected by metabolomic analysis of each brain region was 707. At a significance level of p<0.05(5% of all detected metabolites), 35 differences between groups can be expected by random chance. A number of significantly different metabolites were identified when the following comparisons were made: (1) young naïve mice compared to aged naïve mice, (2) stroked mice at each time point compared to aged naïve mice, (3) ipsilateral hemispheres compared to contralateral hemispheres at matched time points, (4) stroked mice compared to sham mice at matched time points, (5) contralateral hemispheres at each time point compared to aged naïve mice, (6) sham mice at each time point compared to aged naïve mice, and 7) contralateral hemispheres compared to sham mice at matched time points.

There were 190 statistically significant changes by Welch's two-sample t-test between the samples from the young and aged naïve mice, while there were 202 changes at 1 dpi when compared to aged naïve brains, 153 changes at 1 dpi when compared to time point-matched contralateral hemispheres, and 167 changes at 1 dpi when compared to time point-matched samples from sham mice.

### Metabolic Changes Caused by Stroke

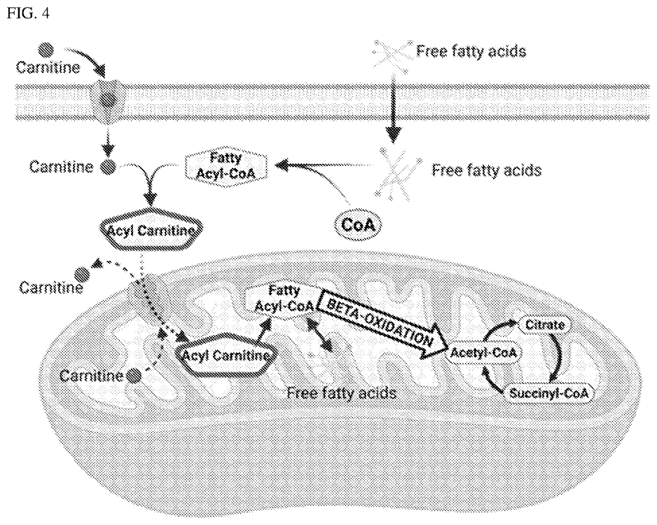

There was a prominent alteration in the abundance of long chain fatty acids in the ipsilateral hemispheres of stroked mice compared to age-matched naïve controls. By 2 weeks after stroke, the levels of multiple long chain fatty acids were significantly increased, peaking at 4 weeks after stroke; however, the levels were indistinguishable from naïve controls by 12 weeks after stroke (FIG. 2). In addition, levels of acyl carnitines, such as pentadecanoylcarnitine (C15), oleoylcarnitine (C18:1), docosapentaenoylcarnitine (C22:5n3) or 3-hydroxyhexanoylcarnitine (1), were increased by stroke (FIG. 3); however, acyl carnitine levels were largely normalized by 8 weeks after stroke. The modification of fatty acids to acyl carnitines is essential for transfer across the inner mitochondrial membrane and subsequent β-oxidation (FIG. 4). These results indicate that there is an increase in the β-oxidation of fatty acids for at least 4 weeks in the ipsilateral hemisphere, which is likely due to the catabolismof myelin lipid debris in the infarct and areas of secondary neurodegeneration in the weeks after stroke (Becktel D A, Zbesko J C, Frye J B, et al. Repeated Administration of 2-Hydroxypropyl-β-Cyclodextrin (HPβCD) Attenuates the Chronic Inflammatory Response to Experimental Stroke. J Neurosci 2022; 42:325-348; Chung A G, Frye J B, Zbesko J C, et al. Liquefaction of the Brain following Stroke Shares a Similar Molecular and Morphological Profile with Atherosclerosis and Mediates Secondary Neurodegeneration in an Osteopontin-Dependent Mechanism. eNeuro 2018; 5: ENEURO.0076-18.2018).

To localize the observed alterations in fatty acid metabolism and to identify the cell types responsible for these changes, a spatial transcriptomic experiment was conducted with aged mice at 4 weeks post stroke. Tissue sections were stained with antibodies against Tmem 119 for microglia/macrophages, GFAP for astrocytes and NeuN for neurons (FIG. 5A-B), and cell type specific RNA was collected from cortex, hippocampus, and thalamus from both ipsi-and contralateral hemispheres. Cell enrichment for the RNA isolated using each barcoded antibody was confirmed by measuring expression levels of genes associated with astrocytes and microglia/macrophages (FIG. 5C), and astrocytes and neurons (FIG. 5D) for each RNA pool from each brain area.

In the neurons, genes associated with glycolysis were more highly expressed than genes associated with fatty acid oxidation and there were no major changes in the ipsilateral versus contralateral hemisphere (FIG. 5E). In astrocytes, genes associated with fatty acid oxidation were more highly expressed than genes associated with glycolysis, and the strongest change was a decrease in the expression of genes related to fatty acid oxidation in the ipsilateral thalamus (FIG. 5F). In microglia/macrophages, genes associated with glycolysis were more highly expressed than genes associated with beta oxidation. However, in the ipsilateral thalamus and lesion there was a pronounced increase in the expression of beta oxidation associated genes, and a corresponding decrease in the expression of genes related to glycolysis (FIG. 5G). These findings indicate that when beta oxidation levels are altered in the brain after stroke they localize to microglia and macrophages in the lesion and area of axonal degeneration.

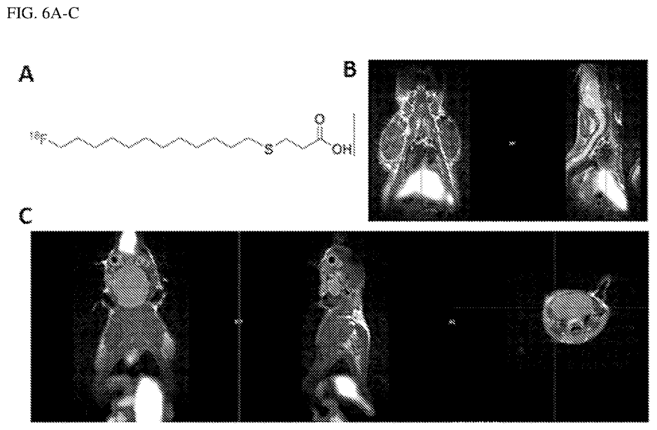

An 18F-palmitate derivative was delivered to naïve mice and mice two weeks after they had undergone stroke surgery to determine if fatty acid β-oxidation was detectable within the stroke-damaged tissue through PET MRI. In naïve mice, the tracer emphasized fatty acid β-oxidation in heart tissue, but not in brain tissue. However, two weeks post-stroke, the tracer revealed an increase in fatty acid β-oxidation in the infarcted tissue of the brain (FIG. 6).

All publications, patents, patent applications and accession numbers mentioned in the above specification are herein incorporated by reference in their entirety. Although the invention has been described in connection with specific embodiments, it should be understood that the invention as claimed should not be unduly limited to such specific embodiments. Indeed, various modifications and variations of the described compositions and methods of the invention will be apparent to those of ordinary skill in the art and are intended to be within the scope of the following claims.

1. A method of identifying foam cells in the brain after neurological damage, comprising:
a) imaging the central nervous system (CNS) of a subject that has had neurological damage for the presence of brain fatty acid oxidation (BFAO) using radiopharmaceutical fatty acid metabolic probes and imaging; and
b) identifying said subject as having foam cells in the CNS when the presence of BFAO is identified.

2. A method of treating a subject that has had neurological damage, comprising:
a) imaging the CNS of a subject that has had neurological damage for the presence of brain fatty acid oxidation (BFAO) using radiopharmaceutical fatty acid metabolic probes and imaging; and
b) administering a lipid chelator when the presence of BFAO is identified.

3. (canceled)

4. A method of monitoring a treatment in a subject that has had neurological damage, comprising:
a) imaging the CNS of a subject that has had neurological damage for the presence of brain fatty acid oxidation (BFAO) using radiopharmaceutical fatty acid metabolic probes and imaging;
b) administering a lipid chelator when the presence of BFAO is identified; and
c) repeating said assaying step after said administering.

5. The method of claim 1, wherein said neurological damage is due to an injury or neurological disorder.

6. The method of claim 5, wherein said injury or neurological damage is selected from the group consisting of ): Stroke, traumatic brain injury (TBI), spinal cord injury, multiple sclerosis (MS), progressive multifocal leukoencephalopathy (PML), leukodystrophies, Niemann-Pick disease, cerebral small vessel disease (SVD), chronic traumatic encephalopathy (CTE), neuromyelitis optica (NMO), hypoxic-ischemic injury, infections affecting the CNS like Lyme disease and HIV, radiation leukoencephalopathy, age-related white matter changes, drugs and toxins leading to leukoencephalopathy, vascular dementia, and Binswanger's disease.

7. The method of claim 1, wherein said neurological damage causes degenerating white matter in the brain of said subject.

8. The method of claim 1, wherein said imaging comprises magnetic resonance imaging (MRI) or Positron emission tomography (PET).

9. The method of claim 1, further comprising assaying a sample from said subject for the level of one or more markers selected from the group consisting of acyl carnitines and long chain fatty acids in a sample from said subject.

10. The method of claim 9, wherein said sample is blood, a blood product, cells, or tissue.

11. The method of claim 1, wherein the level of one or more of said markers is increased in microglia in areas of degenerating white matter.

12. The method of claim 6, wherein said stroke is an ischemic stroke.

13. The method of claim 1, wherein said assaying is performed at a time period of one or more of 24 hours, 1 week, 2 weeks, 4 weeks, 7 weeks, and 6 months after neurological damage.

14. The method of claim 13, wherein said assaying is repeated at an interval of 1 day, 1 week, one month, or one year.

15. The method of claim 1, wherein said radiopharmaceutical fatty acid metabolic probe is selected from the group consisting of ¹¹C-palmitate (CPA), ¹⁸F-palmitate derivatives, 16-¹⁸F-fluorohexadecanoic acid (FHDA), 17-¹⁸F-fluoroheptadecanoic acid (FHA), 15-(4-(2-¹⁸F fluoroethoxy)phenyl)pentadecanoic acid (F7), ¹²³I-iodophenylpentadecanoic acid (IPPA), β-methyl-1-¹¹C-heptadecanoic acid (β-Me-HA), 3-Methyl-17-¹⁸F-fluoroheptadecanoic acid (3MFHA), 5-methyl-17-¹⁸F-fluoroheptadecanoic acid (5MFHA), β-methyl-15-¹²³I-iodophenylpentadecanoic acid (β-MeIPPA), 9-^{123m}Te-telluraheptadecanoic acid (9-TeHA), 14-¹⁸F-fluoro-6-thia-heptadecanoicacid (FTHA), 16-¹⁸F-fluoro-4-thia-hexadecanoic acid (FTP), 18-¹⁸F-fluoro-4-thia-oleic acid (FTO), and trans-9(RS)-¹⁸F-fluoro-3,4(RS, RS)-methyleneheptadecanoic acid (¹⁸F-FCPHA).

16. The method of claim 1, wherein said cyclodextrin is 2-Hydroxypropyl-b-cyclodextrin (HPbCD).

17. The method of claim 1, wherein said lipid chelator is cyclodextrin.

18. The method of claim 1, wherein said CNS is brain or spinal cord.

19-20. (canceled)
